SEQUENCE LISTING (10 sequences)

SEQ ID NO: 1 - RNA, 20 nt, Mus musculus
  source 1..20: mol_type = other RNA
gacgctgagccggggcccca

SEQ ID NO: 2 - RNA, 20 nt, Mus musculus
  source 1..20: mol_type = other RNA
tggggccccggctcagcgtc

SEQ ID NO: 3 - RNA, 20 nt, Mus musculus
  source 1..20: mol_type = other RNA
tgcgctcctcgtggagcaga

SEQ ID NO: 4 - RNA, 20 nt, Mus musculus
  source 1..20: mol_type = other RNA
cgccgcccttctgctccacg

SEQ ID NO: 5 - RNA, 20 nt, Mus musculus
  source 1..20: mol_type = other RNA
ctgctcggctgcgctcctcg

SEQ ID NO: 6 - RNA, 20 nt, Mus musculus
  source 1..20: mol_type = other RNA
gttgcaggtttccctactcc

SEQ ID NO: 7 - RNA, 20 nt, Mus musculus
  source 1..20: mol_type = other RNA
caccagcctggcctggagta

SEQ ID NO: 8 - RNA, 20 nt, Mus musculus
  source 1..20: mol_type = other RNA
gcaccagcctggcctggagt

SEQ ID NO: 9 - RNA, 20 nt, Mus musculus
  source 1..20: mol_type = other RNA
aggtttccctactccaggcc

SEQ ID NO: 10 - RNA, 20 nt, Mus musculus
  source 1..20: mol_type = other RNA
ggcggtcctcagaagccagg

(19) United States
(12) Patent Application Publication
(10) Pub. No.: US 2026/0262614 A1
(43) Pub. Date: Sep. 10, 2026

(54) CEREBRAL MICROBLEED ANIMAL MODEL AND PREPARATION METHOD THEREFOR

(71) Applicant: AJOU UNIVERSITY INDUSTRY-ACADEMIC COOPERATION FOUNDATION, Suwon-si (KR)

(72) Inventors: Byung Gon Kim, Suwon-si (KR); Hyunmi Kim, Suwon-si (KR)

(21) Appl. No.: 19/680,985

(22) Filed: May 18, 2026

Related U.S. Application Data
(63) Continuation of application No. PCT/KR2024/013480, filed on Sep. 6, 2024.

(30) Foreign Application Priority Data
Nov. 23, 2023 (KR) 10-2023-0164161
Feb. 16, 2024 (KR) 10-2024-0022517

(51) Int. Cl. A01K 67/0276; C12N 9/22; C12N 15/11; C12N 15/86

(52) U.S. Cl. CPC A01K 67/0276; C12N 9/226; C12N 15/111; C12N 15/86; A01K 2217/075; A01K 2227/105; A01K 2267/0375; C12N 2310/20; C12N 2750/14143; C12N 2750/14145

(57) ABSTRACT

Provided is a cerebral microbleed animal model, wherein the cerebral microbleed is induced by a Col4a1-targeting gRNA (guide RNA) packaged in AAV-BR1.

### INCORPORATION OF SEQUENCE LISTING

This application contains a sequence listing submitted in Computer Readable Form (CRF). The CRF file containing the sequence listing entitled “PK6128126-SequenceListing.xml”, which was created on May 15, 2026, and is 9,830 bytes in size. The information in the sequence listing is incorporated herein by reference in its entirety.

### FIELD

The present disclosure relates to a cerebral microbleed animal model through cerebral blood vessel-specific editing of a COL4A1 gene.

### DESCRIPTION OF THE RELATED ART

Among cerebral pathological phenomena caused by abnormalities of cerebral small vessels, cerebral microbleed (CMB) is known to occur due to microhemorrhage and hemosiderin deposition caused by damage to cerebral small vessels. With advances in brain magnetic resonance imaging techniques, cerebral microbleeds of less than several millimeters are being increasingly commonly detected in the elderly population. When stroke occurs in patients having cerebral microbleeds, the prognosis is known to be much worse, and it is also known that the risk of intracerebral hemorrhage is increased and the possibility of occurrence of intracerebral hemorrhage as a side effect caused by antithrombotic and anticoagulant therapy is increased. In addition, cerebral microbleeds are frequently found in cerebral amyloid angiopathy (CAA), which is a vascular abnormality observed in patients with Alzheimer's disease, and reports have shown that the number of cerebral microbleeds is correlated with the degree of cognitive decline. Accordingly, the possibility that cerebral microbleeds may be a factor in cognitive decline caused by dementia has also been raised.

Therefore, although there is a need for treatment for preventing cerebral microbleeds or inhibiting progression thereof, the molecular and cellular mechanisms by which cerebral microbleeds occur have not yet been clearly identified, and thus therapeutic targets for inhibiting the same remain unclear.

In addition, establishment of an appropriate animal model is essential for elucidating the pathophysiological mechanisms of cerebral microbleeds. However, to date, no animal model exhibiting cerebral microbleeds similar to those observed in human MRI images has been reported.

Accordingly, there is a need to develop a cerebral microbleed animal model for elucidating the pathophysiological mechanisms of cerebral microbleeds and for developing therapeutic agents for the prevention or treatment of cerebral microbleeds.

Korean Patent Publication No. 10-2023-0077849, which is a background art of the present disclosure, relates to a method for detecting cerebral microbleeds and a learning method for detecting cerebral microbleeds.

### DESCRIPTION

### Technical Problem

The present disclosure has been made to solve the problems of the above-described conventional art, and provides a cerebral microbleed animal model exhibiting imaging and pathological findings very similar to those of human cerebral microbleeds by selectively depleting COL4A1 in cerebral microvascular endothelial cells.

The present disclosure also provides a method for preparing the cerebral microbleed animal model.

The present disclosure also provides a method for screening a therapeutic agent for cerebral microbleed using the cerebral microbleed animal model.

However, the technical problems to be achieved by the embodiments of the present disclosure are not limited to the technical problems described above, and there may be other technical problems.

### Means for Solving the Problem

As a technical means for achieving the above-described technical problems, a first aspect of the present disclosure provides a cerebral microbleed animal model, wherein a cerebral microbleed is induced by a Col4a1-targeting gRNA (guide RNA) packaged in AAV-BR1.

According to one embodiment of the present disclosure, expression of a COL4A1 gene may be inhibited by the Col4a1-targeting gRNA, but is not limited thereto.

According to one embodiment of the present disclosure, elasticity of a blood-brain barrier (BBB) may be reduced by inhibition of expression of the COL4A1 gene, but is not limited thereto.

According to one embodiment of the present disclosure, the cerebral microbleed may be induced by reduction in elasticity of the blood-brain barrier (BBB), but is not limited thereto.

According to one embodiment of the present disclosure, the Col4a1-targeting gRNA may be delivered specifically to cerebral vascular endothelial cells by the AAV-BR1, but is not limited thereto.

According to one embodiment of the present disclosure, the Col4a1-targeting gRNA (guide RNA) may comprise a nucleotide sequence of any one of SEQ ID NOs: 1 to 9, but is not limited thereto.

In addition, a second aspect of the present disclosure provides a method for preparing a cerebral microbleed animal model, comprising inducing a cerebral microbleed by injecting a Col4a1-targeting gRNA (guide RNA) packaged in AAV-BR1 into a CRISPR-Cas9 expressing transgenic animal.

According to one embodiment of the present disclosure, expression of a COL4A1 gene may be inhibited by the Col4a1-targeting gRNA, but is not limited thereto.

According to one embodiment of the present disclosure, elasticity of a blood-brain barrier (BBB) may be reduced by inhibition of expression of the COL4A1 gene, but is not limited thereto.

According to one embodiment of the present disclosure, the cerebral microbleed may be induced by reduction in elasticity of the blood-brain barrier (BBB), but is not limited thereto.

According to one embodiment of the present disclosure, the Col4a1-targeting gRNA may be delivered specifically to cerebral vascular endothelial cells by the AAV-BR1, but is not limited thereto.

According to one embodiment of the present disclosure, the Col4a1-targeting gRNA may comprise a nucleotide sequence of any one of SEQ ID NOs: 1 to 9, but is not limited thereto.

In addition, a third aspect of the present disclosure provides a method for screening a therapeutic agent for cerebral microbleed, comprising: administering a candidate substance for a therapeutic agent for cerebral microbleed to the cerebral microbleed animal model according to the first aspect of the present disclosure; and comparing the animal model administered with the candidate substance with a control group not administered with the candidate substance, and determining the candidate substance as a therapeutic agent for cerebral microbleed when the cerebral microbleed is reduced by the candidate substance.

The aforementioned means for solving the problem are merely exemplary and should not be construed as intending to limit the present disclosure. In addition to the exemplary embodiments described above, further embodiments may exist in the drawings and the detailed description of the invention.

### Effect of the Invention

The cerebral microbleed animal model according to the present disclosure may be used as a cerebral microbleed model by packaging a Col4a1-targeting gRNA specifically targeting a Col4a1 gene in an AAV-BR1 virus directed specifically to cerebral vascular endothelial cells and injecting the same into an animal, thereby delivering the Col4a1-targeting gRNA only to cerebral blood vessels to edit the Col4a1 gene specifically in the cerebral blood vessels, and consequently weakening elasticity of cerebral blood vessels of the animal to induce microbleeding in the brain.

In addition, the cerebral microbleed animal model according to the present disclosure may be utilized for developing therapeutic agents for various brain diseases accompanied by cerebral microbleed symptoms, and may be utilized for developing biomarkers capable of early detection of cerebral microbleed.

However, the effects obtainable from the present disclosure are not limited to the effects described above, and other effects may also be present.

### BRIEF DESCRIPTION OF THE DRAWING

FIG. 1A shows results of confirming, by an indel assay, whether a Col4a1 gene was edited by delivering Col4a1-targeting gRNA sequences according to one experimental example of the present disclosure into mouse NIH3T3 cells, and FIG. 1B shows results of confirming a Col4a1 mRNA level.

FIG. 2A shows results of confirming expression levels of Col4a1 mRNA and protein by real-time PCR after treatment with a Col4a1-targeting gRNA according to one experimental example of the present disclosure, and FIG. 2B shows results confirmed by a western blot experiment.

FIG. 3A is a schematic diagram of an experiment for confirming Col4a1 gene editing and inhibition of COL4A1 expression in cerebral vascular endothelial cells by injecting a Col4a1-targeting gRNA packaged in AAV-BR1 according to one experimental example of the present disclosure into a CRISPR-Cas9 transgenic animal, FIG. 3B shows indel results, and FIG. 3C shows results of measuring an out-of-frame ratio of the edited Col4a1 gene.

FIG. 4A shows results of confirming, by real-time PCR, whether Col4a1 mRNA and protein were reduced in cerebral blood vessels by culturing primary cerebral vascular endothelial cells from cerebral blood vessels of an animal three weeks after injecting a Col4a1-targeting gRNA packaged in AAV-BR1 according to one experimental example of the present disclosure into a CRISPR-Cas9 transgenic animal, FIG. 4B shows results confirmed by a western blot experiment, and FIG. 4C shows results obtained by staining brain tissue sections and observing the same with a confocal microscope.

FIG. 5 shows magnetic resonance imaging results of a brain of a CRISPR-Cas9 transgenic animal injected with a Col4a1-targeting gRNA packaged in AAV-BR1 according to one experimental example of the present disclosure.

FIG. 6A shows results of cerebral microbleeds on a brain surface according to injection concentrations of a Col4a1-targeting gRNA packaged in AAV-BR1 according to one experimental example of the present disclosure, FIG. 6B shows results of cerebral microbleeds in brain sections, and FIG. 6C shows results of measuring, by region, lesions caused by cerebral microbleeds in brain tissue sections of the animal three months after injection.

FIG. 7A shows results of confirming cerebral microbleeds in brain tissue sections using Prussian blue staining three months after injecting a Col4a1-targeting gRNA packaged in AAV-BR1 according to one experimental example of the present disclosure into animals at various concentrations, FIG. 7B shows results of observing hemosiderin granules in regions stained with Prussian blue, and FIG. 7C shows results of performing immunostaining using anti-Ferritin Light/Heavy chain antibodies.

FIG. 8A shows results of measuring short-term memory using a Y-maze test from 0 months to 6 months after injecting a Col4a1-targeting gRNA packaged in AAV-BR1 according to one experimental example of the present disclosure into animals, FIG. 8B shows results of measuring cognitive function and memory ability using a novel object recognition test, and FIG. 8C shows results of performing a neurobehavioral test for examining balance, limb coordination, muscle strength, and the like using a rotarod test.

### DETAILED DESCRIPTION

Hereinafter, examples of the present disclosure will be described in detail so as to be easily implemented by those skilled in the art, with reference to the accompanying drawings. However, the present disclosure may be embodied in many different forms and are not limited to the examples to be described herein. In addition, parts not related with the description have been omitted in order to clearly describe the present disclosure in the drawings and throughout the present specification, like reference numerals designate like elements.

Further, throughout this specification, when a certain part is “connected” with the other part, it is meant that the certain part may be “directly connected” with the other part and “electrically connected” with the other part with another element interposed therebetween.

Throughout the present specification, it will be understood that when a certain member is located “on”, “above”, “at the top of”, “under”, “below”, and “at the bottom of” the other member, a certain member is in contact with the other member and another member may also be present between the two members.

Throughout the specification, a case where a part “includes” an element will be understood to imply the inclusion of stated elements but not the exclusion of any other elements unless explicitly described to the contrary.

The terms “about”, “substantially”, and the like to be used in the specification are used as a numerical value or a value close to the numerical value when inherent manufacturing and material tolerances are presented in the stated meaning, and used to prevent an unscrupulous infringer from unfairly using disclosed contents in which precise or absolute numerical values are mentioned to help in the understanding of the present disclosure. Throughout the present specification, the term of “step to” or “step of” does not mean “step for”.

Throughout the present specification, the term “combinations thereof” included in the expression of the Markush form means one or more mixtures or combinations selected from the group consisting of components described in the expression of the Markush form, and means to include at least one selected from the group consisting of the components.

Throughout the present specification, “A and/or B” means “A or B, or A and B”.

Hereinafter, the cerebral microbleed animal model of the present disclosure will be described in detail with reference to embodiments, examples, and drawings. However, the present disclosure is not limited to these embodiments, examples, and drawings.

As a technical means for achieving the above-described technical problems, a first aspect of the present disclosure provides a cerebral microbleed animal model, wherein a cerebral microbleed is induced by a Col4a1-targeting gRNA (guide RNA) packaged in AAV-BR1.

The blood-brain barrier (BBB) serves to protect the brain from various pathogens or toxins of pathogens while allowing only selective permeability between the brain and blood vessels. The blood-brain barrier (BBB) has a structure in which blood vessels are very tightly surrounded by cerebral vascular endothelial cells, cerebral vascular pericytes, and astrocytes, and a basement membrane surrounds the outer surface of the endothelial cells. Among these components, COL4A1 protein, which is one subtype of collagen, constitutes a major component.

The cerebral microbleed animal model according to the present disclosure may be used as a cerebral microbleed model by packaging a Col4a1-targeting gRNA specifically targeting a Col4a1 gene in an AAV-BR1 virus directed specifically to cerebral vascular endothelial cells and injecting the same into an animal, thereby delivering the Col4a1-targeting gRNA only to cerebral blood vessels to edit the Col4a1 gene specifically in the cerebral blood vessels, and consequently weakening elasticity of cerebral blood vessels of the animal to induce microbleeding in the brain.

In addition, the cerebral microbleed animal model according to the present disclosure may be utilized for developing therapeutic agents for various brain diseases accompanied by cerebral microbleed symptoms, and may be utilized for developing biomarkers capable of early detection of cerebral microbleed.

According to one embodiment of the present disclosure, expression of a COL4A1 gene may be inhibited by the Col4a1-targeting gRNA, but is not limited thereto.

According to one embodiment of the present disclosure, elasticity of the blood-brain barrier (BBB) may be reduced by inhibition of expression of the COL4A1 gene, but is not limited thereto.

According to one embodiment of the present disclosure, the cerebral microbleed may be induced by reduction in elasticity of the blood-brain barrier (BBB), but is not limited thereto.

According to one embodiment of the present disclosure, the Col4a1-targeting gRNA may be delivered specifically to cerebral vascular endothelial cells by the AAV-BR1, but is not limited thereto.

The animal model according to the present disclosure can inhibit expression of the COL4A1 gene specifically in cerebral vascular endothelial cells of an animal by packaging a Col4a1-targeting gRNA in an AAV-BR1 virus directed specifically to cerebral vascular endothelial cells and injecting the same into the animal, and cerebral microbleed may thereby be induced.

According to one embodiment of the present disclosure, the Col4a1-targeting gRNA (guide RNA) may comprise a nucleotide sequence of any one of SEQ ID NOs: 1 to 9, but is not limited thereto.

In the animal model according to the present disclosure, an sgRNA (single guide RNA) sequence capable of specifically acting on a mouse Col4a1 gene without acting on other genes may be designed and injected into an animal, thereby inhibiting expression of the COL4A1 gene in cerebral vascular endothelial cells of the animal to induce cerebral microbleed.

In addition, a second aspect of the present disclosure provides a method for preparing a cerebral microbleed animal model, comprising inducing a cerebral microbleed by injecting a Col4a1-targeting gRNA (guide RNA) packaged in AAV-BR1 into a CRISPR-Cas9 expressing transgenic animal.

With respect to the method for preparing a cerebral microbleed animal model according to the second aspect of the present disclosure, detailed descriptions of the parts overlapping with the first aspect of the present disclosure have been omitted. However, even if the descriptions are omitted, the contents described in the first aspect of the present disclosure may be equally applied to the second aspect of the present disclosure.

According to one embodiment of the present disclosure, expression of a COL4A1 gene may be inhibited by the Col4a1-targeting gRNA, but is not limited thereto.

According to one embodiment of the present disclosure, elasticity of the blood-brain barrier (BBB) may be reduced by inhibition of expression of the COL4A1 gene, but is not limited thereto.

According to one embodiment of the present disclosure, the cerebral microbleed may be induced by reduction in elasticity of the blood-brain barrier (BBB), but is not limited thereto.

According to one embodiment of the present disclosure, the Col4a1-targeting gRNA may be delivered specifically to cerebral vascular endothelial cells by the AAV-BR1, but is not limited thereto.

According to one embodiment of the present disclosure, the Col4a1-targeting gRNA may comprise a nucleotide sequence of any one of SEQ ID NOs: 1 to 9, but is not limited thereto.

In addition, a third aspect of the present disclosure provides a method for screening a therapeutic agent for cerebral microbleed, comprising: administering a candidate substance for a therapeutic agent for cerebral microbleed to the cerebral microbleed animal model according to the first aspect of the present disclosure; and comparing the animal model administered with the candidate substance with a control group not administered with the candidate substance, and determining the candidate substance as a therapeutic agent for cerebral microbleed when the cerebral microbleed is reduced by the candidate substance.

With respect to the method for screening a therapeutic agent for cerebral microbleed according to the third aspect of the present disclosure, detailed descriptions of the parts overlapping with the first aspect and/or the second aspect of the present disclosure have been omitted. However, even if the descriptions are omitted, the contents described in the first aspect and/or the second aspect of the present disclosure may be equally applied to the third aspect of the present disclosure.

Hereinafter, the present invention will be described in more detail through examples. However, the following examples are provided only for illustrative purposes and are not intended to limit the scope of the present disclosure.

### [Experimental Methods]

### INDEL Assay

A target site was amplified using Phusion High-Fidelity DNA Polymerase PCR Polymerase (NEB) with 30 ng of genomic DNA (gDNA). Using IDT for Illumina DNA/RNA UD

Indexes (Illumina, San Diego, CA, USA), the amplified target-site amplicon was further amplified to generate a deep sequencing library. Paired-end sequencing was performed using an Illumina NextSeq system, and indel frequency was calculated at “http://www.rgenome.net/.”

### RNA Extraction & Real-Time PCR

RNA was extracted from primary endothelial cells cultured from the brain of a transgenic animal. The extraction method was as follows.

Primary endothelial cells were mixed with Trizol, chloroform was added thereto at a ratio of 4:1, followed by invert mixing, and the mixture was allowed to stand at room temperature for a short time. When the layers were separated, only the supernatant was separated by centrifugation at 13,000 rpm for 15 minutes at 4° C. The separated supernatant was invert-mixed with isopropanol at a ratio of 1:1 and allowed to stand at room temperature for 1 hour to precipitate RNA. After 1 hour, the mixture was centrifuged at 13,000 rpm for 15 minutes at 4° C., the remaining supernatant was completely removed while leaving only a pellet, and the pellet was washed with 75% ice-cold EtOH. The pellet was then thoroughly dried and dissolved in 0.1% DEPC-DW.

RNA was synthesized into cDNA according to a protocol of cDNA synthesis master mix (Cell Safe, Korea). Col4a1 expression was analyzed by real-time PCR, and primer sequences were as follows.

SEQ ID NO: mouse Col4a1
[F]
5′-ATG GCT TGC CTG GAG AGA TAG G-3′
[R]
5′-TGG TTG CCC TTT GAG TCC TGG A-3′
SEQ ID NO: mouse Gapdh
[F]
5′-CAT CAC TGC CAC CCA GAA GAC TG-3′
[R]
5′-ATG CCA GTG AGC TTC CCG TTC AG-3′

### Western Blot

For protein extraction, primary endothelial cells cultured from the brain of a transgenic animal were dissociated in a lysis reagent. Thereafter, the resulting lysate was centrifuged at 15,000 g for 5 minutes at 4° C., and only the supernatant was separated and then incubated at 95° C. for 5 minutes. The tissue samples were loaded onto a Mini-PROTEAN® TGX™ Gel (Bio-Rad, California, USA), and electrophoresis was performed in an electrophoresis apparatus at 100 V for 1 hour and 30 minutes. After electrophoresis was completed, the gel was transferred to an NC membrane at 30 V for 18 hours at a low temperature. After transfer, the NC membrane was incubated with an anti-COL4A1 primary antibody (ab6586, Abcam, 1:1000) diluted in 5% skim milk at 4° C. for 16 hours. Thereafter, the NC membrane was incubated with an HRP-conjugated anti-rabbit IgG secondary antibody (Cell Signaling, 1:5000) at room temperature for 50 minutes, and then detected using an X-ray film.

### H&E Staining & Prussian Blue Staining

Tissue samples for tissue staining to confirm histology were prepared using tissues fixed by perfusion with PBS and 4% PFA after experimental animals were anesthetized with ketamine. After perfusion, tissues collected from the experimental animals were treated with a 10% sucrose solution and a 30% sucrose solution at 4° C. The sucrose-treated tissues were sectioned to a thickness of 30 μm using a frozen microtome. The sectioned tissues were attached to tissue slides, and tissue staining was performed. Before staining, the tissue sections were subjected to dehydration-rehydration and then stained with a Prussian blue staining solution or a Hematoxylin & Eosin staining solution. The stained tissue slides were imaged and analyzed using a slide scanner (Axioscan Z1, Zeiss, Jena, Germany).

### Tissue Immunostaining (Ferritin Light & Heavy Chain, COL4A1, and Iba1 Staining)

Tissue samples for tissue immunostaining were prepared using tissues fixed by perfusion with PBS and 4% PFA after experimental animals were anesthetized with ketamine. After perfusion, tissues collected from the experimental animals were treated with a 10% sucrose solution and a 30% sucrose solution at 4° C. The sucrose-treated tissues were sectioned to a thickness of 30 μm using a frozen microtome. The sectioned tissues were attached to tissue slides, and tissue immunostaining was performed. Before immunostaining, the tissue slides were incubated at room temperature for 30 minutes with a solution containing 0.3% Triton X-100 and 10% NGS. As primary antibodies for tissue immunostaining, an anti-Ferritin Light chain antibody (ab69090, Abcam, 1:250), an anti-Ferritin Heavy chain antibody (ab65080, Abcam, 1:100), and an anti-COL4A1 antibody (ab6586, Abcam, 1:100) were used. The antibody labeling process was performed by incubating the tissue slides with the primary antibodies diluted in 10% NGS at 4° C. for 16 hours, followed by incubation with secondary antibodies at room temperature for 2 hours. After antibody incubation was completed, the tissue slides were fixed with a mounting medium and imaged using a microscope. Tissue slides stained with the COL4A1 antibody were imaged using a Zeiss LSM900 microscope (Jena, Germany). Tissue slides stained with the anti-Ferritin Light & Heavy chain antibodies were imaged and analyzed using a slide scanner.

### Animal Magnetic Resonance Imaging

For imaging, experimental animals were subjected to respiratory anesthesia with 1.5% isoflurane. As MRI equipment, a high-field 9.4 T animal magnetic resonance imaging apparatus was used, and T2-weighted images were obtained according to a coronal T2-weighted RARE sequence. (TR/TE=3300/60 ms, echo train length=4, FOV=14×11 mm², matrix size=256×200)

### Y-Maze Test

As an experiment for measuring short-term memory, the Y-maze test was performed by the following method in order to evaluate the ability to perform sequential behavior. The measurement apparatus was composed of three arms, each arm having a length of 32.5 cm, a width of 7 cm, and a height of 15 cm, and the angle at which the three arms met was 120°. The apparatus was made of white polyvinyl plastic, and the experiment was performed after designating the three arms as A, B, and C, respectively. An experimental animal was placed in the apparatus for 10 minutes, and the number of entries into each arm was counted when the tail of the experimental animal entered the arm. In addition, when the experimental animal entered each arm sequentially, 1 point was assigned as an actual alternation. Alternation behavior was defined as entry into all three arms without overlap, and spontaneous alternation was calculated according to Equation 1 below.

$$Alternation triplet ( \% ) = [ Alternation / ( total number of entries - 2 ) ] \times 100 \qquad [ Equation 1 ]$$

### Novel Object Recognition Test

Mice were placed in an open-field box (40 cm×40 cm×40 cm) made of acrylic for 10 minutes to allow habituation and stabilization in the box. On the second day (training day), two different objects (objects A and B) made of acrylic were placed in the box at a predetermined distance from each other, and the mice were trained to freely explore objects A and B for 10 minutes. Thereafter, one of objects A and B was replaced with a novel object (object C), and the mice were allowed to explore again for 10 minutes. The exploration time for each object (A and C or B and C) was quantified, and preference for the object was expressed as a ratio of exploration time according to Equation 2 below.

$$Preference ( \% ) = [ C / ( A + C ) ] \times 100 \qquad [ Equation 2 ]$$

### Rotarod Test

In the rotarod test, an experimental mouse was placed on a rotarod apparatus, and the apparatus was operated by starting at a speed of 4 rpm and gradually increasing the speed to 40 rpm. When the treadmill was rotated, the time (sec) until the experimental mouse lost balance and fell to the floor and the average speed were measured. A sensor was provided on the floor so that the time could be automatically measured when the experimental mouse touched the floor.

### [Example 1] Col4a1_E01-#01

In order to edit a target gene sequence by a gRNA delivered into cells, a mouse Col4a1-target sgRNA packaged in an AAV-BR1 virus was intravenously injected through the orbital vein of a transgenic animal expressing CRISPR-Cas9 nuclease (6J.129 (Cg)-Gt (ROSA) 26Sortm1.1 (CAG-cas9*,-EGFP) Fezh/J).

The sequence of the injected Col4a1-target sgRNA was as follows.

SEQ ID NO: 1:
5′-GACGCTGAGCCGGGGCCCCA-3′

### [Example 2] Col4a1_E01-#02

The same method as in Example 1 was performed, except that a Col4a1-target sgRNA comprising a nucleotide sequence of SEQ ID NO: 2 below was used.

SEQ ID NO: 2:
5′-TGGGGCCCCGGCTCAGCGTC-3′

### [Example 3] Col4a1_E01-#03

The same method as in Example 1 was performed, except that a Col4a1-target sgRNA comprising a nucleotide sequence of SEQ ID NO: 3 below was used.

SEQ ID NO: 3:
5′-TGCGCTCCTCGTGGAGCAGA-3′

### [Example 4] Col4a1_E01-#04

The same method as in Example 1 was performed, except that a Col4a1-target sgRNA comprising a nucleotide sequence of SEQ ID NO: 4 below was used.

SEQ ID NO: 4:
5′-CGCCGCCCUUCUGCUCCACG-3′

### [Example 5] Col4a1_E01-#05

The same method as in Example 1 was performed, except that a Col4a1-target sgRNA comprising a nucleotide sequence of SEQ ID NO: 5 below was used.

SEQ ID NO: 5:
5′-CTGCTCGGCTGCGCTCCTCG-3′

### [Example 6] Col4a1_E21-#01

The same method as in Example 1 was performed, except that a Col4a1-target sgRNA comprising a nucleotide sequence of SEQ ID NO: 6 below was used.

SEQ ID NO: 6:
5′-GTTGCAGGTTTCCCTACTCC-3′

### [Example 7] Col4a1_E21-#02

The same method as in Example 1 was performed, except that a Col4a1-target sgRNA comprising a nucleotide sequence of SEQ ID NO: 7 below was used.

SEQ ID NO: 7:
5′-CACCAGCCTGGCCTGGAGTA-3′

### [Example 8] Col4a1_E21-#03

The same method as in Example 1 was performed, except that a Col4a1-target sgRNA comprising a nucleotide sequence of SEQ ID NO: 8 below was used.

SEQ ID NO: 8:
5′-GCACCAGCCTGGCCTGGAGT-3′

### [Example 9] Col4a1_E21-#04

The same method as in Example 1 was performed, except that a Col4a1-target sgRNA comprising a nucleotide sequence of SEQ ID NO: 9 below was used.

SEQ ID NO: 9:
5′-AGGTTTCCCTACTCCAGGCC-3′

### [Comparative Example 1] Rosa26-target sgRNA

The same method as in Example 1 was performed, except that a mouse Rosa26-target sgRNA sequence was used.

The injected mouse Rosa26-target sgRNA sequence was as follows.

SEQ ID NO: 10:
5′-GGCGGUCCUCAGAAGCCAGG-3′

### [Experimental Example 1] Design and Selection of gRNA Sequences for Efficient Editing of Col4a1 Gene

In order to efficiently edit the Col4a1 gene, CRISPR-Cas9 gRNA sequences were designed and screened.

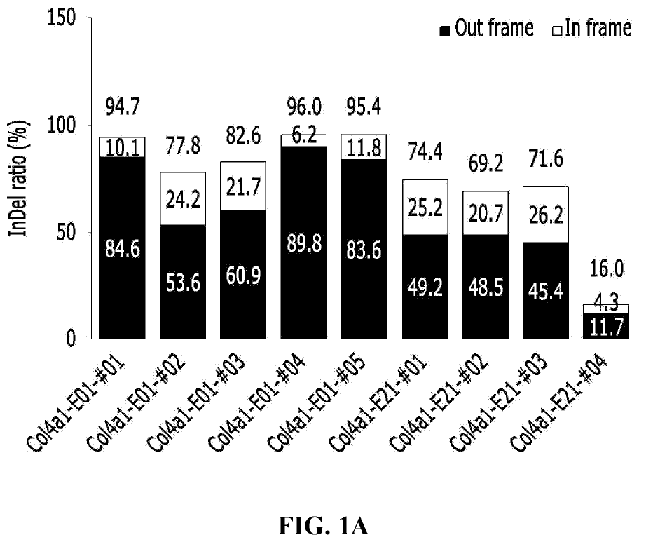

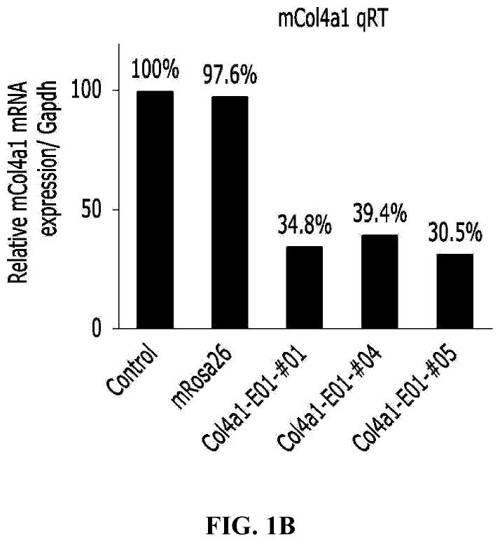

FIG. 1A shows results of confirming, by an indel assay, whether the Col4a1 gene was edited by delivering Col4a1-targeting gRNA sequences according to one experimental example of the present disclosure into mouse NIH3T3 cells, and FIG. 1B shows results of confirming a Col4a1 mRNA level.

Referring to FIGS. 1A-1B, it was confirmed that the gRNA sequence used in Example 4 (Col4a1_E01-#04) edited the Col4a1 gene most efficiently. Accordingly, in the experimental examples described below, experiments were performed using the gRNA sequence of Example 4.

### [Experimental Example 2] Observation of Inhibition of COL4A1 Expression by Selected Col4a1 E01-#04 in Primary Cerebral Vascular Endothelial Cells

Primary cerebral vascular endothelial cells obtained from cerebral blood vessels of CRISPR-Cas9 transgenic animals were cultured, and the Col4a1_E01-#04 sequence was packaged in a lentivirus. The virus was delivered into cells at concentrations of 5×10⁵, 2.5×10⁶, and 5×10⁶ TU/ml, and then whether Col4a1 mRNA and protein were efficiently reduced was confirmed by real-time PCR and western blot experiments.

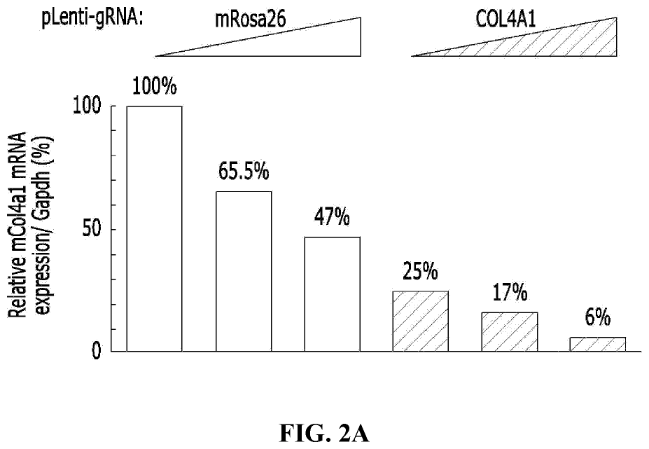

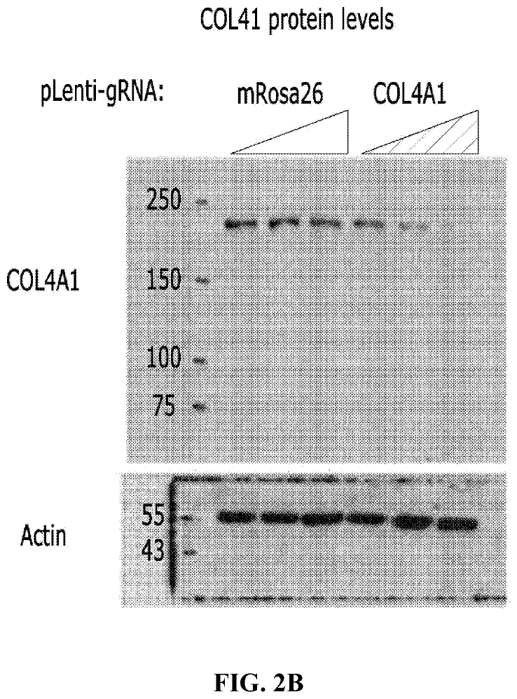

FIG. 2A shows results of confirming expression levels of Col4a1 mRNA and protein by real-time PCR after treatment with the Col4a1-targeting gRNA according to one experimental example of the present disclosure, and FIG. 2B shows results confirmed by a western blot experiment.

Referring to FIGS. 2A-2B, clear inhibition of COL4A1 expression was observed in the primary cerebral vascular endothelial cells by treatment with the Col4a1-targeting gRNA.

Accordingly, in the experimental examples described below, experiments were performed using the gRNA sequence of Example 4 packaged in an AAV-BR1 virus.

### [Experimental Example 3] Confirmation of Col4a1 Gene Editing and Inhibition of COL4A1 Expression in Actual Cerebral Vascular Endothelial Cells by Injection of AAV-BR1-Col4a1 E01-#04 Virus in CRISPR-Cas9 Transgenic Animals

Using an AAV-BR1 system directed specifically to cerebral blood vessels, Col4a1_E01-#04 was packaged in and produced as an AAV-BR1 virus. The virus was delivered to cerebral blood vessels by intravenous injection through the orbital vein at concentrations of 0.5E+11 GC, 1E+11 GC, and 2E+11 GC scale. Three weeks after injection, primary cerebral vascular endothelial cells were cultured from cerebral blood vessels of the animals, and whether the Col4a1 gene was actually edited in the cerebral vascular endothelial cells was confirmed by an indel assay.

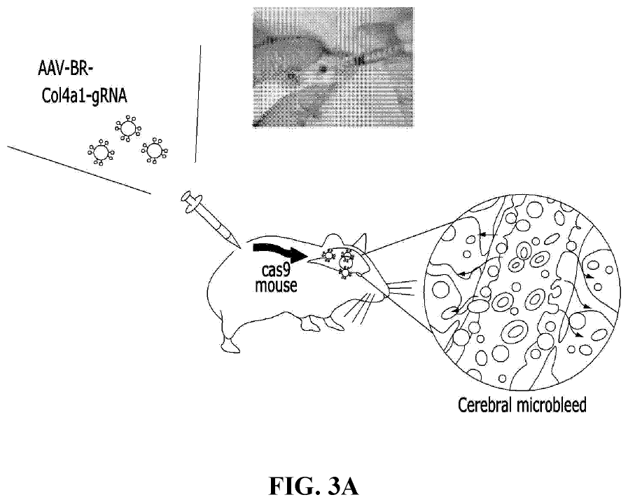

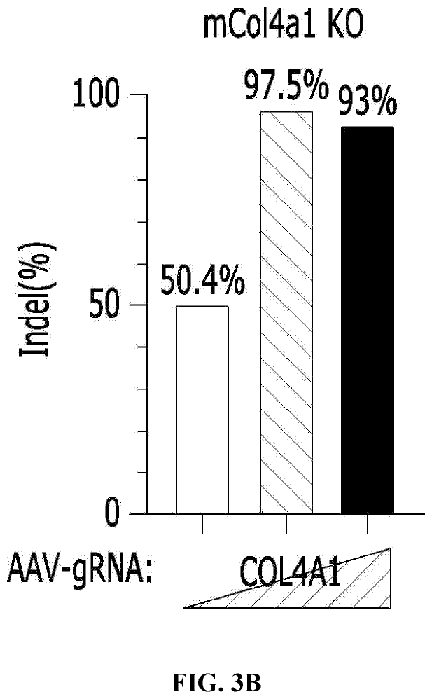

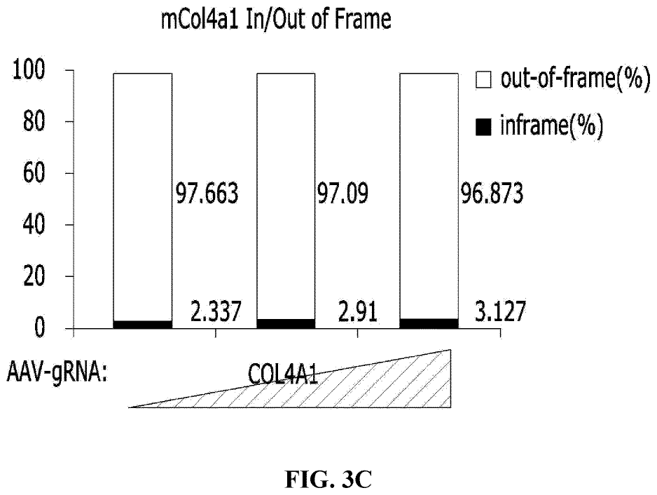

FIG. 3A is a schematic diagram of an experiment for confirming Col4a1 gene editing and inhibition of COL4A1 expression in cerebral vascular endothelial cells by injecting a Col4a1-targeting gRNA packaged in AAV-BR1 according to one experimental example of the present disclosure into CRISPR-Cas9 transgenic animals, FIG. 3B shows indel results, and FIG. 3C shows results of measuring an out-of-frame ratio of the edited Col4a1 gene.

From the indel results, when AAV-BR1-Col4a1_E01-#04 was injected at a scale of 1E+11 GC or more, Col4a1 gene editing of 95% or more was confirmed (FIG. 3B). Among the deleted 95% of genes, the out-of-frame ratio was confirmed to be 97% or more, thereby confirming that almost all Col4a1 genes were completely edited (FIG. 3C).

Meanwhile, three weeks after virus injection, primary cerebral vascular endothelial cells were cultured from cerebral blood vessels of the animals, and whether Col4a1 mRNA and protein were reduced in the cerebral blood vessels was confirmed by real-time PCR and western blot experiments. In addition, reduction of COL4A1 expression in brain tissue sections was confirmed by staining and observation with a confocal microscope.

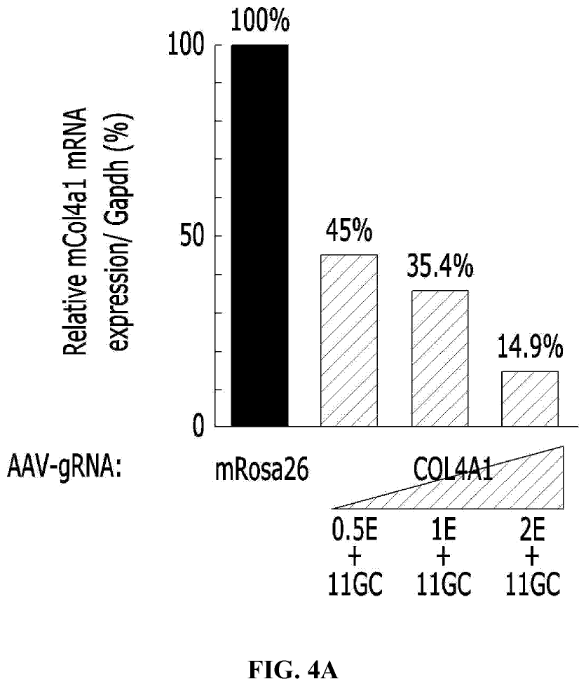

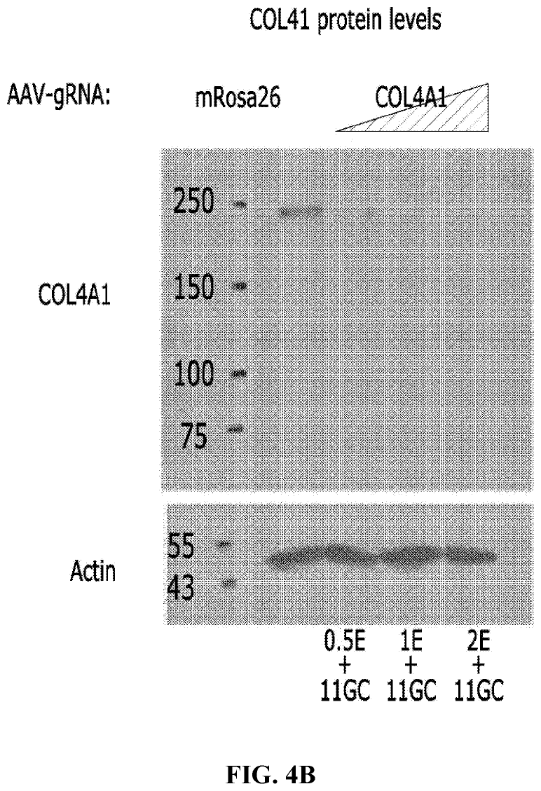

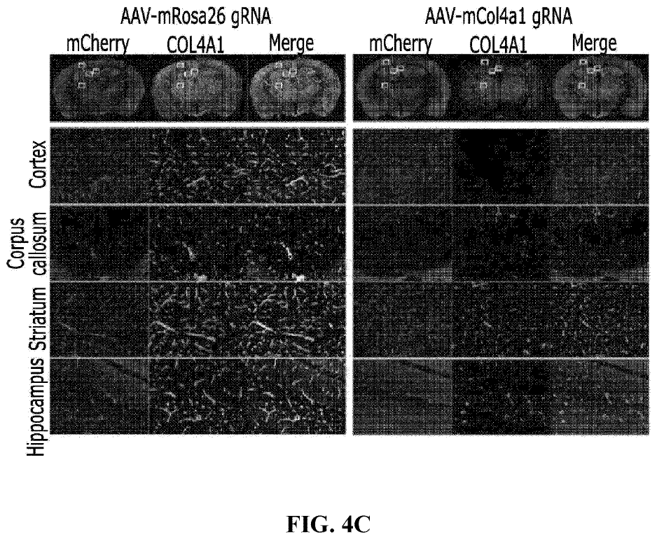

FIG. 4A shows results of confirming, by real-time PCR, whether Col4a1 mRNA and protein were reduced in cerebral blood vessels by culturing primary cerebral vascular endothelial cells from cerebral blood vessels of animals three weeks after injecting a Col4a1-targeting gRNA packaged in AAV-BR1 according to one experimental example of the present disclosure into CRISPR-Cas9 transgenic animals, FIG. 4B shows results confirmed by a western blot experiment, and FIG. 4C shows results obtained by staining brain tissue sections and observing the same with a confocal microscope.

Referring to FIGS. 4A-4C, it was confirmed that COL4A1 expression was almost reduced when the virus was injected at a scale of 1E+11 GC or more in the CRISPR-Cas9 transgenic animals.

### [Experimental Example 4] Magnetic Resonance Imaging of Cerebral Microbleeds Caused by Injection of AAV-BR1-Col4a1_E01-#04 Virus in CRISPR-Cas9 Transgenic Animals

Three months after injection of the AAV-BR1-Col4a1_E01-#04 virus (2E+11 GC scale), cerebral microbleeds in the brains of animals were confirmed by magnetic resonance imaging.

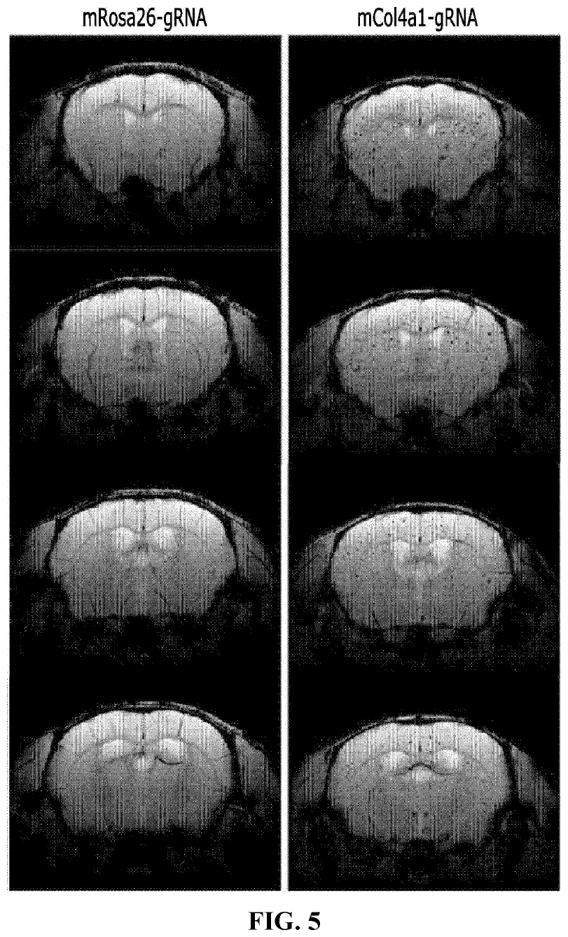

FIG. 5 shows magnetic resonance imaging results of a brain of a CRISPR-Cas9 transgenic animal injected with a Col4a1-targeting gRNA packaged in AAV-BR1 according to one experimental example of the present disclosure.

Referring to FIG. 5, microbleeds having a form similar to microbleeds observed in humans were confirmed in the CRISPR-Cas9 transgenic animal injected with the Col4a1-targeting gRNA packaged in AAV-BR1. Accordingly, in the experimental examples described below, the microbleeds observed in the CRISPR-Cas9 transgenic animal injected with the Col4a1-targeting gRNA packaged in AAV-BR1 were verified through various experiments.

### [Experimental Example 5] Confirmation of Microbleeds on Brain Surface and in Brain Sections Caused by Injection of AAV-BR1-Col4a1_E01-#04 Virus in CRISPR-Cas9 Transgenic Animals

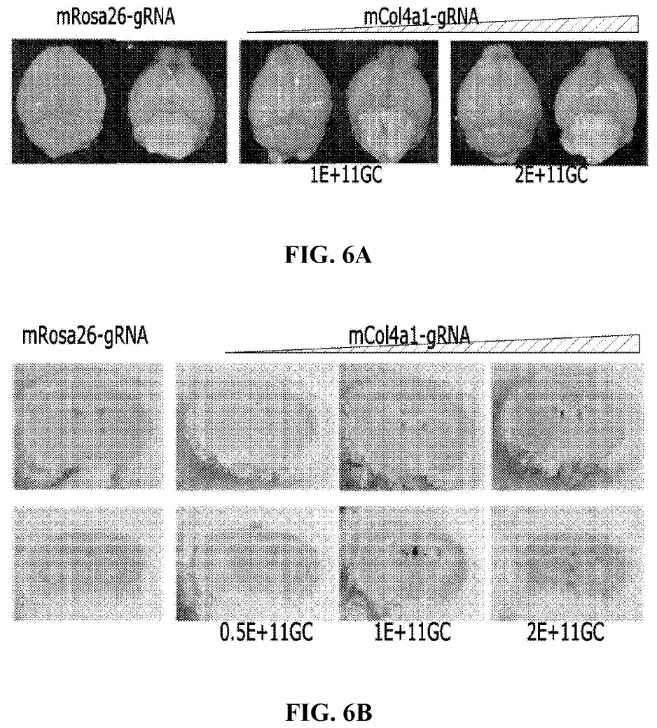

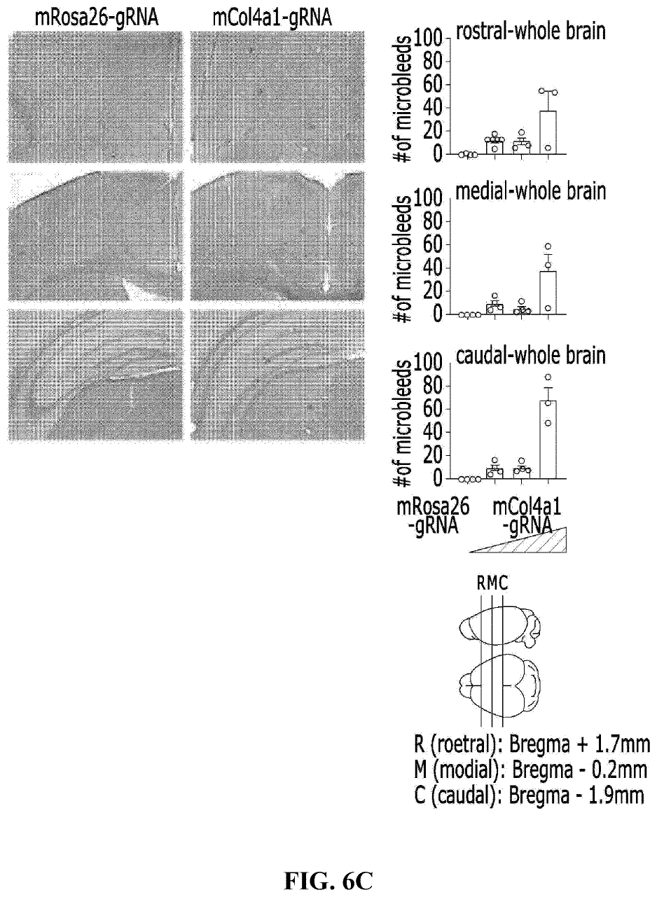

FIG. 6A shows results of cerebral microbleeds on a brain surface according to injection concentrations of a Col4a1-targeting gRNA packaged in AAV-BR1 according to one experimental example of the present disclosure, FIG. 6B shows results of cerebral microbleeds in brain sections, and FIG. 6C shows results of measuring, by region, lesions caused by cerebral microbleeds in brain tissue sections of the animal three months after injection.

Referring to FIG. 6A, the AAV-BR1-Col4a1_E01-#04 virus was delivered to cerebral blood vessels by intravenous injection through the orbital vein at concentrations of 1E+11 GC and 2E+11 GC scale, and traces of microbleeds were confirmed on the brain surface of the animals three months after virus injection.

Referring to FIG. 6B, it was confirmed that microbleeds were observed in brain sections three months after the AAV-BR1-Col4a1_E01-#04 virus was intravenously injected through the orbital vein at concentrations of 0.5E+11 GC, 1E+11 GC, and 2E+11 GC scale.

Referring to FIG. 6C, three months after injection of the AAV-BR1-Col4a1 E01-#04 virus, lesions caused by cerebral microbleeds in brain tissue sections of the animals were measured by region through hematoxylin & eosin staining, and as a result, it was confirmed that regions where lesions appeared were increased.

### [Experimental Example 6] Tissue Staining for Confirming Cerebral Microbleeds Caused by Injection of AAV-BR1-Col4a1 E01-#04 Virus in CRISPR-Cas9 Transgenic Animals

The AAV-BR1-Col4a1 E01-#04 virus was delivered to cerebral blood vessels by intravenous injection through the orbital vein at concentrations of 0.5E+11 GC, 1E+11 GC, and 2E+11 GC scale. Three months after virus injection, Prussian blue staining was performed to confirm cerebral microbleeds in brain tissue sections of the animals. Three months after injection of the AAV-BR1-Col4a1 E01-#04 virus (2E+11 GC scale), hemosiderin granules were observed in regions stained with Prussian blue. In addition, in order to further confirm cerebral microbleeds, an immunostaining method using anti-Ferritin Light/Heavy chain antibodies was performed.

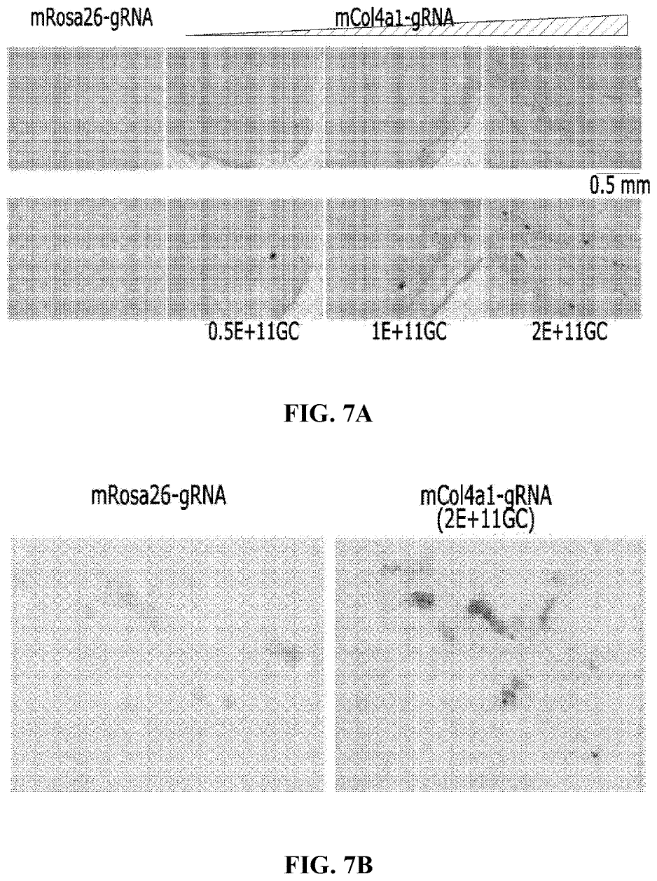

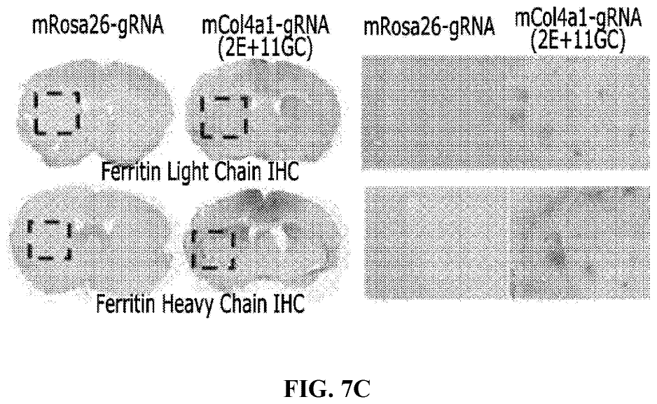

FIG. 7A shows results of confirming cerebral microbleeds in brain tissue sections using Prussian blue staining three months after injecting a Col4a1-targeting gRNA packaged in AAV-BR1 according to one experimental example of the present disclosure into animals at various concentrations, FIG. 7B shows results of observing hemosiderin granules in regions stained with Prussian blue, and FIG. 7C shows results of performing immunostaining using anti-Ferritin Light/Heavy chain antibodies.

Referring to FIGS. 7A-7C, cerebral microbleeds were confirmed through various staining methods for verifying microbleeds in brain sections caused by injection of the AAV-BR1-Col4a1 E01-#04 virus in the CRISPR-Cas9 transgenic animals.

### [Experimental Example 7] Observation of Cognitive Impairment and Motor Function Reduction in Mice Injected with AAV-BR1-mCol4a1-target gRNA

In order to observe cognitive impairment and motor function reduction in mice injected with AAV-BR1-mCol4a1-target gRNA, a Y-maze test, a novel object recognition test, and a rotarod test were performed.

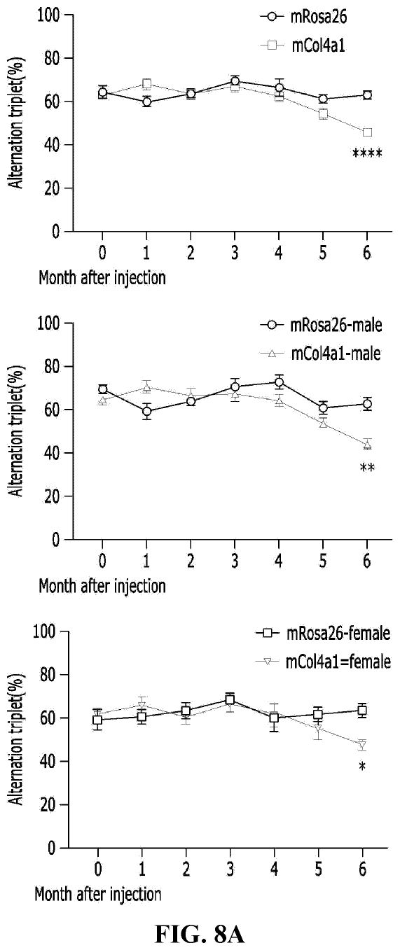

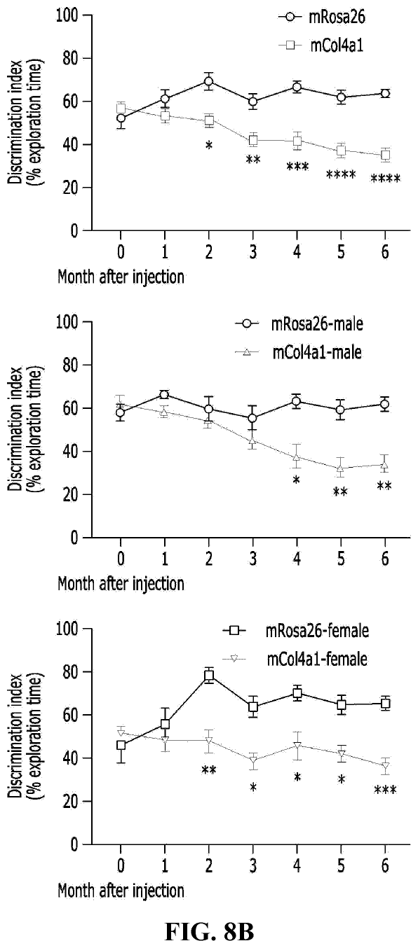

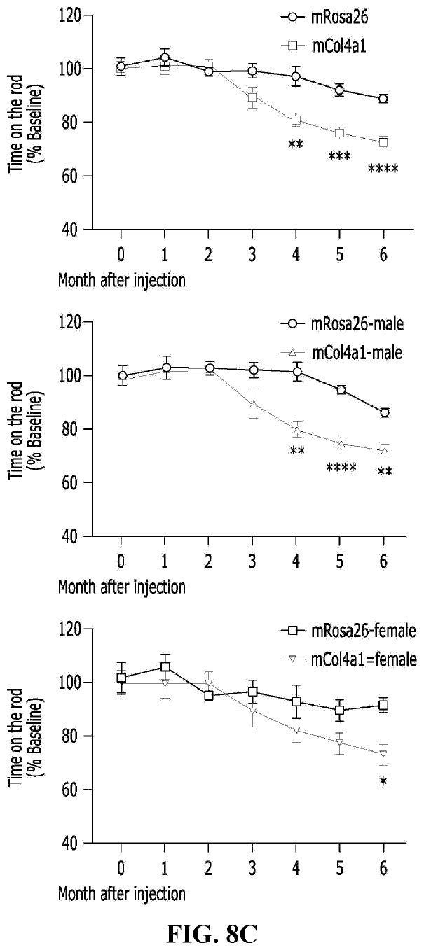

FIG. 8A shows results of measuring short-term memory from 0 months to 6 months by a Y-maze test after injecting a Col4a1-targeting gRNA packaged in AAV-BR1 according to one experimental example of the present disclosure into animals, FIG. 8B shows results of measuring cognitive function and memory ability by a novel object recognition test, and FIG. 8C shows results of performing a neurobehavioral test capable of examining balance, limb coordination, muscle strength, and the like by a rotarod test.

The upper graph of FIG. 8A shows statistics obtained by combining male and female transgenic animals, the middle graph shows statistics obtained from male transgenic animals under the same conditions, and the lower graph shows statistics obtained from female transgenic animals under the same conditions. Through these results, it was confirmed that short-term memory was reduced in CRISPR-Cas9 transgenic animals at 6 months after injection of the Col4a1-targeting gRNA packaged in AAV-BR1 by comparing the number of successive entries into three different arms in alternation relative to the total number of entries into the Y-maze.

The upper graph of FIG. 8B shows statistics obtained by combining male and female transgenic animals, and shows a statistically significant reduction effect from 2 months after injection. The middle graph shows statistics obtained from male transgenic animals under the same conditions, and shows a statistically significant reduction effect from 4 months after injection. The lower graph shows statistics obtained from female transgenic animals under the same conditions, and shows a statistically significant reduction effect from 2 months after injection. Through these results, it was confirmed that cognitive function and memory ability were reduced in the CRISPR-Cas 9 transgenic animals injected with the Col4a1-targeting gRNA packaged in AAV-BR1 by measuring the exploration time for a novel object.

The upper graph of FIG. 8C shows statistics obtained by combining male and female transgenic animals, and shows a statistically significant reduction effect from 4 months after injection. The middle graph shows statistics obtained from male transgenic animals under the same conditions, and shows a statistically significant reduction effect from 4 months after injection. The lower graph shows statistics obtained from female transgenic animals under the same conditions, and shows a statistically significant reduction effect at 6 months after injection. Through these results, it was confirmed that balance, limb coordination, muscle strength, and the like were reduced in the CRISPR-Cas9 transgenic animals injected with the Col4a1-targeting gRNA packaged in AAV-BR1 by checking the falling time from the treadmill.

The aforementioned description of the present disclosure is to be exemplified, and it will be understood by those skilled in the art that the present disclosure may be easily modified in other detailed forms without changing the technical spirit or required features of the present disclosure. Therefore, it should be appreciated that the examples described above are illustrative in all aspects and are not restricted. For example, each component described as a singular form may be implemented in a distributed manner, and components described as being distributed may also be implemented in a combined form.

The scope of the present disclosure is represented by appended claims to be described below rather than the detailed description, and it is to be interpreted that the meaning and scope of the claims and all the changes or modified forms derived from the equivalents thereof come within the scope of the present disclosure.

1. A cerebral microbleed animal model, wherein the cerebral microbleed is induced by Col4a1-targeting gRNA (guide RNA) packaged in AAV-BR1.

2. The cerebral microbleed animal model of claim 1,
wherein expression of a COL4A1 gene is inhibited by the Col4a1-targeting gRNA.

3. The cerebral microbleed animal model of claim 2,
wherein elasticity of a blood-brain barrier (BBB) is reduced by inhibition of expression of the COL4A1 gene.

4. The cerebral microbleed animal model of claim 3,
wherein the cerebral microbleed is induced by reduction in elasticity of the blood-brain barrier (BBB).

5. The cerebral microbleed animal model of claim 1,
wherein the Col4a1-targeting gRNA is delivered specifically to cerebral vascular endothelial cells by the AAV-BR1.

6. The cerebral microbleed animal model of claim 1,
wherein the Col4a1-targeting gRNA (guide RNA) comprises a nucleotide sequence of any one of SEQ ID NOs: 1 to 9.

7. A method for preparing a cerebral microbleed animal model, comprising inducing cerebral microbleed by injecting a Col4a1-targeting gRNA (guide RNA) packaged in AAV-BR1 into a CRISPR-Cas9 expressing transgenic animal.

8. The method for preparing a cerebral microbleed animal model of claim 7,
wherein expression of a COL4A1 gene is inhibited by the Col4a1-targeting gRNA.

9. The method for preparing a cerebral microbleed animal model of claim 8,
wherein elasticity of a blood-brain barrier (BBB) is reduced by inhibition of expression of the COL4A1 gene.

10. The method for preparing a cerebral microbleed animal model of claim 9,
wherein the cerebral microbleed is induced by reduction in elasticity of the blood-brain barrier (BBB).

11. The method for preparing a cerebral microbleed animal model of claim 7,
wherein the Col4a1-targeting gRNA is delivered specifically to cerebral vascular endothelial cells by the AAV-BR1.

12. The method for preparing a cerebral microbleed animal model of claim 7, wherein the Col4a1-targeting gRNA comprises a nucleotide sequence of any one of SEQ ID NOs: 1 to 9.

13. A method for screening a therapeutic agent for cerebral microbleed, comprising:
administering a candidate substance for a therapeutic agent for cerebral microbleed to the cerebral microbleed animal model according to claim 1; and
comparing the animal model administered with the candidate substance with a control group not administered with the candidate substance, and determining the candidate substance as a therapeutic agent for cerebral microbleed when the cerebral microbleed is reduced by the candidate substance.
